(19) United States
(12) Patent Application Publication
(10) Pub. No.: US 2026/0294670 A1
(43) Pub. Date: Oct. 1, 2026

(54) Pants device, Excrement Collecting Assembly, and Nursing Machine

(71) Applicant: SUZHOU ALTON ELECTRICAL & MECHANICAL INDUSTRY CO., LTD., Suzhou, Jiangsu (CN)

(72) Inventors: Weidong LU, West Chicago, IL (US); Qiang SONG, Suzhou, Jiangsu (CN); Dengke LEI, Suzhou, Jiangsu (CN); Wei LI, Suzhou, Jiangsu (CN); Jingtao PAN, Suzhou, Jiangsu (CN)

(73) Assignee: SUZHOU ALTON ELECTRICAL & MECHANICAL INDUSTRY CO., LTD., Suzhou, Jiangsu (CN)

(21) Appl. No.: 18/881,596

(22) PCT Filed: Jul. 4, 2023
(86) PCT No.: PCT/CN2023/105669

§ 371 Date: Jan. 6, 2025

(30) Foreign Application Priority Data
Jul. 5, 2022 (CN) 202210793924.6
Sep. 20, 2022 (CN) 202211145747.7
Sep. 20, 2022 (CN) 202222491928.7
Dec. 17, 2022 (CN) 202223407970.2

(51) Int. Cl. A61F 5/441; A61F 5/442

(52) U.S. Cl. CPC A61F 5/441; A61F 5/442

(57) ABSTRACT

A pants device, an excrement collecting assembly, and a nursing machine are provided. The pants device is configured to be worn on a human body, and includes a connecting frame and a crotch opening for human excretion. The connecting frame is located at a crotch of the pants device. The connecting frame surrounds at least a part of the crotch opening. The connecting frame is provided with an air inlet, an air outlet, and an airflow channel. The airflow channel communicates the air inlet with the air outlet. The connecting frame of the pants device is provided with the air inlet, the airflow channel and the air outlet which are in communication with each other, so that the air at the air inlet may be supplemented into the air outlet when the air pressure at the air outlet is lower than the air pressure at the air inlet.

### CROSS-REFERENCE TO RELATED APPLICATION

The disclosure is a National Stage Filing of the PCT International Application No. PCT/CN2023/105669 filed on Jul. 4, 2023, which claims priority to the Chinese patent application No. 202210793924.6, filed with the Patent Office of the People's Republic of China on Jul. 5, 2022, the Chinese patent application No. 202222491928.7, filed with the Patent Office of the People's Republic of China on Sep. 20, 2022, the Chinese patent application No. 202211145747.7, filed with the Patent Office of the People's Republic of China on Sep. 20, 2022, and the Chinese patent application No. 202223407970.2, filed with the Patent Office of the People's Republic of China on Dec. 17, 2022. All contents of the above four patent applications are incorporated by reference in the disclosure.

### TECHNICAL FIELD

The disclosure relates to a pants device, an excrement collecting assembly, and a nursing machine, in particular to the improvement of a connecting frame of a crotch of a pants device.

### BACKGROUND

The patent application CN217138536U, published on Feb. 6, 2022, discloses a nursing machine. The nursing machine includes a host, a working head, and a pants device. The pants device includes a pants main part, a connecting frame integrated with the pants main part, and a crotch opening. The connecting frame surrounds at least a part of the crotch opening. The connecting frame is configured for connecting the pants device with the working head. The working head is connected to the connecting frame of the pants device to receive human excreta. The connecting frame includes a first U-shaped part, a second U-shaped part, a left connecting part, and a right connecting part. The first U-shaped part is located at a front lower part of the connecting frame, and the second U-shaped part is located at a rear upper part of the connecting frame. The first U-shaped part, the left connecting part, the second U-shaped part and the right connecting part are sequentially connected to form a closed loop, thereby surrounding the crotch opening (excretion port). The first U-shaped part, the second U-shaped part, the left connecting part and the right connecting part are basically solid. The above connecting frame simply plays a role in external connection. The above connecting frame needs to be further improved to enhance the applicability.

### SUMMARY

Some embodiments of the disclosure provide a pants device, and a connecting frame of the pants device has a path for air circulation, so that the applicability is stronger.

An embodiment of the disclosure provides a pants device for wearing on a human body. The pants device includes a connecting frame and a crotch opening for human excretion. The connecting frame is located at a crotch of the pants device. The connecting frame is configured for connecting the pants device with a working head of a nursing machine. The connecting frame surrounds at least a part of the crotch opening. The connecting frame includes an air inlet, an air outlet, and an airflow channel. The airflow channel communicates the air inlet with the air outlet.

Based on this design, the connecting frame of the pants device is provided with the air inlet, the airflow channel and the air outlet which are in communication with each other, so that the air at the air inlet may be supplemented into the air outlet through the airflow channel when the air pressure at the air outlet is lower than the air pressure at the air inlet. The connecting frame of the pants device has a path for air circulation, so that the applicability is stronger.

Another embodiment of the disclosure provides an excrement collecting assembly, including the above pants device and a working head. The connecting frame is configured for connecting the pants device with the working head. The working head includes an excretion recess for receiving human excreta.

Another embodiment of the disclosure provides a nursing machine, including a host, the above excrement collecting assembly, and a sewage suction pipe for connecting between the working head and the host. The host includes a negative pressure source for sucking excreta. Under the suction effect of the negative pressure source, the air pressure in the excretion recess is lower than the air pressure outside the excrement collecting assembly.

### BRIEF DESCRIPTION OF THE DRAWINGS

FIG. 1 is a three-dimensional view of a pants device in the disclosure;

FIG. 2 is a three-dimensional view of an excrement collecting assembly in the disclosure;

FIG. 3 is an exploded view of a pants device in the disclosure;

FIG. 4 is a three-dimensional view of a working head of an excrement collecting assembly in the disclosure;

FIG. 5 is a three-dimensional view of a front housing of a working head of an excrement collecting assembly in the disclosure;

FIG. 6 is a view in a state where a connecting frame of an excrement collecting assembly and a working head are separated in the disclosure;

FIG. 7 is a horizontal cross-sectional view in a state where a connecting frame of an excrement collecting assembly and a working head are combined together in the disclosure;

FIG. 8 is a three-dimensional view of a connecting frame of a pants device in the disclosure;

FIG. 9 is an exploded view of a connecting frame of a pants device from one perspective in the disclosure;

FIG. 10 is an exploded view of a connecting frame of a pants device from another perspective in the disclosure;

FIG. 11 is a three-dimensional view of a protective net of a pants device in the disclosure;

FIG. 12 is a partially enlarged three-dimensional view of an excrement collecting assembly in the disclosure (an air inlet includes a protective net);

FIG. 13 is a three-dimensional view in a state where another urine hood of a pants device and a protective net are combined together in the disclosure;

FIG. 14 is a three-dimensional view of another urine hood of a pants device in the disclosure;

FIG. 15 is a view in a state where another urine hood and another connecting frame of a pants device and a protective net are separated in the disclosure; and

FIG. 16 is a partial vertical cross-sectional view in a state where another urine hood and another connecting frame of a pants device and a protective net are combined together in the disclosure.

### DETAILED DESCRIPTION OF THE EMBODIMENTS

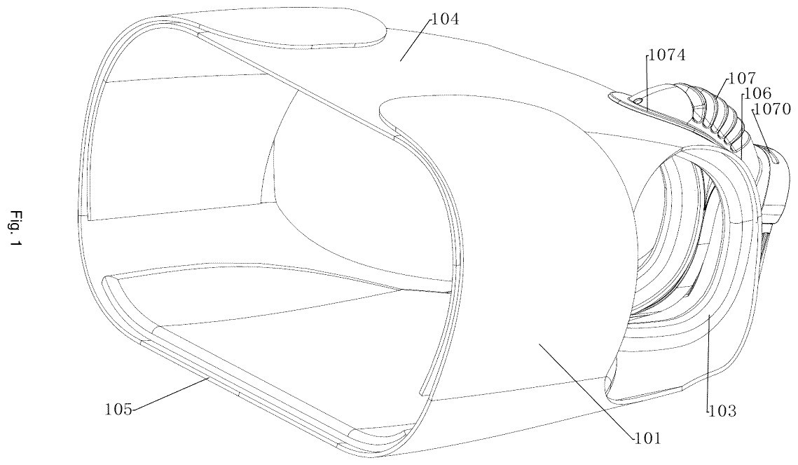

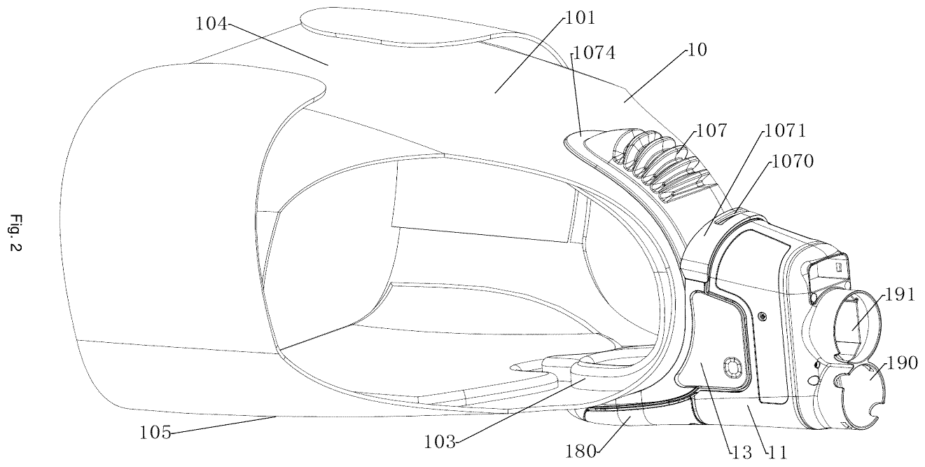

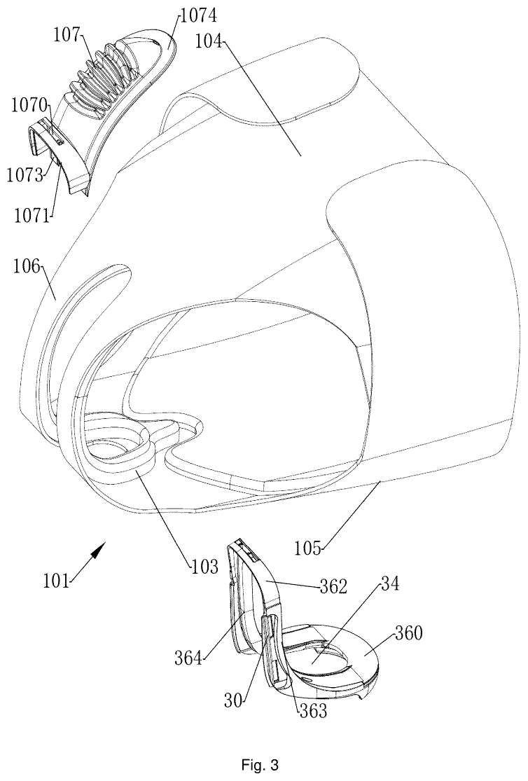

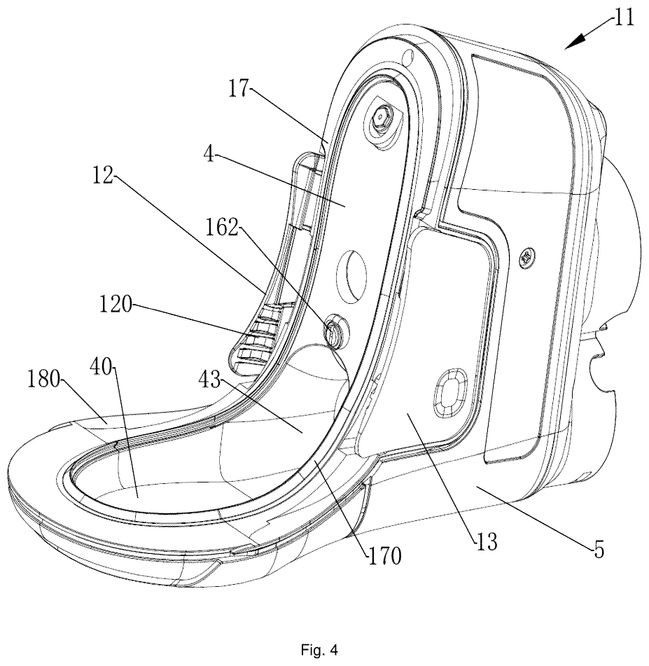

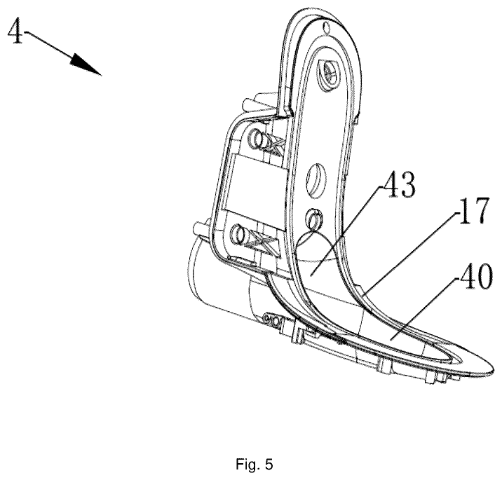

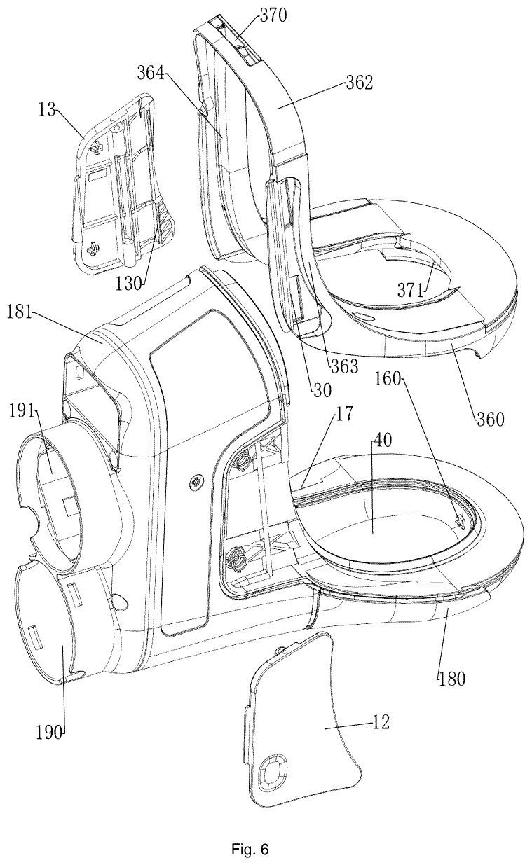

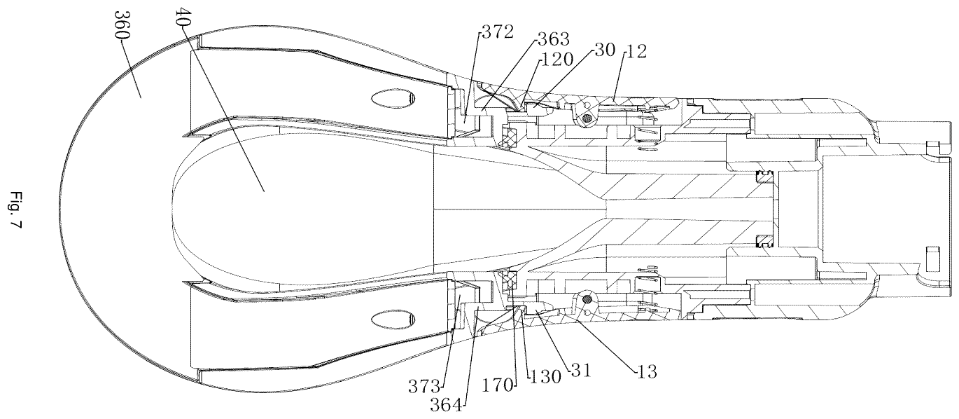

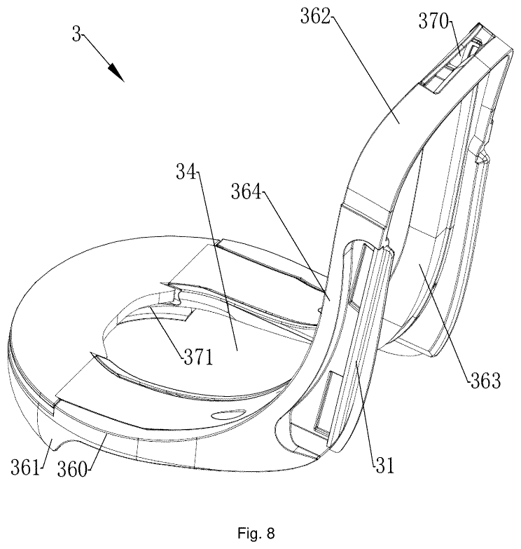

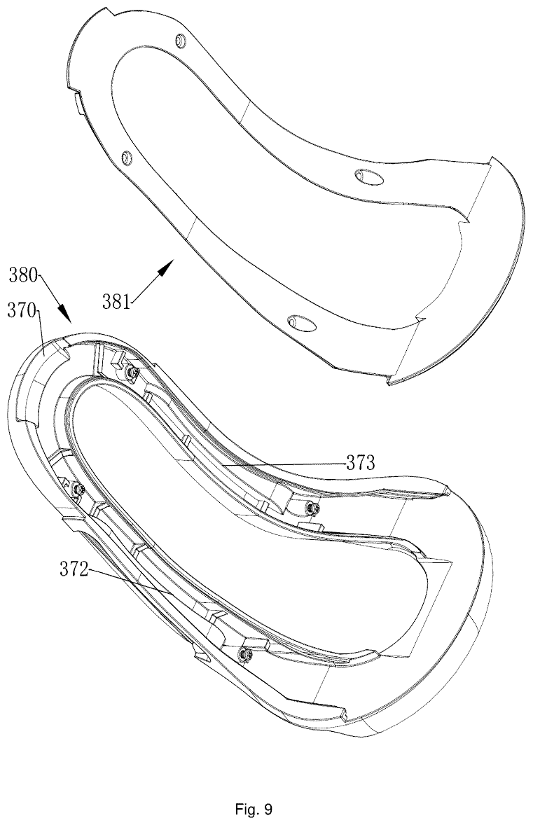

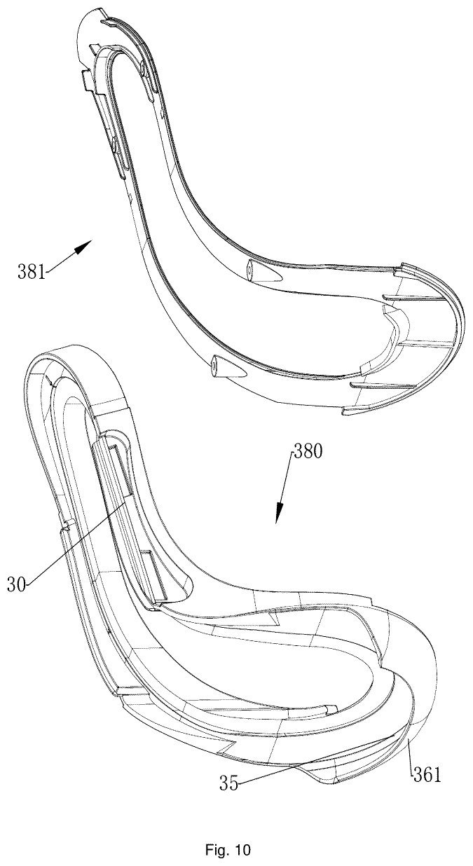

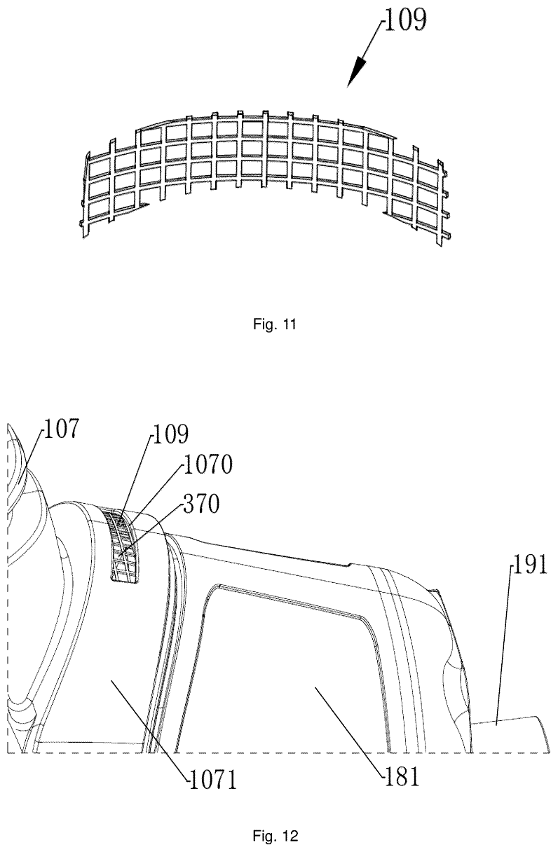

Referring to FIG. 1 to FIG. 12, an excrement collecting assembly of the disclosure includes a pants device 10 and a working head 11. The excrement collecting assembly is configured for a nursing machine. The pants device 10 is configured for wearing on a human body. The pants device 10 includes a connecting frame 3 and a crotch opening 34 for human excretion. The connecting frame 3 is located at a crotch of the pants device 10. The connecting frame 3 surrounds at least a part of the crotch opening 34. The connecting frame 3 includes an air inlet 370, an air outlet 371, and an airflow channel. The airflow channel communicates the air inlet 370 with the air outlet 371. Based on the air inlet 370, the airflow channel and the air outlet 371 which are in communication with each other, the connecting frame 3 of the pants device 10 is provided with an air circulation path, so that the air at the air inlet 370 is able to be supplemented into the air outlet 371 through the airflow channel when the air pressure at the air outlet 371 is lower than the air pressure at the air inlet 370. In summary, the connecting frame 3 of the pants device 10 has a path for delivering air, so that the applicability is stronger. The connecting frame 3 is able to be made of a plastic material.

The connecting frame 3 is configured for butting the pants device 10 with the working head 11 of the nursing machine. During use, the pants device 10 is worn to the human body, and the working head 11 is butted to the connecting frame 3 of the pants device 10 to receive human excreta. The working head 11 is equivalent to being indirectly worn to the human body through the pants device 10. The working head 11 includes an excretion recess 40 for receiving human excreta. In a state where the connecting frame 3 is butted together with the working head 11, the air outlet 371 of the connecting frame 3 is adjacent to the excretion recess 40, thereby facilitating the supplementation of the airflow at the air outlet 371 into the excretion recess 40. After the excrement collecting assembly is worn to the human body, the human body can excrete feces and other excreta into the excretion recess 40 through the crotch opening 34. The connecting frame 3 is annular, and the connecting frame 3 surrounds the crotch opening 34. From another perspective, a frame opening provided and surrounded by the connecting frame 3 forms the crotch opening 34, the air inlet 370 is located at the external periphery of the connecting frame 3, and the air outlet 371 is located at the internal periphery of the connecting frame 3. The air inlet 370 is provided by an upper part of the connecting frame 3, and the air outlet 371 is provided by a lower part of the connecting frame 3.

The connecting frame 3 includes a first U-shaped part 360, a second U-shaped part 362, a left connecting part 363, and a right connecting part 364. The left connecting part 363 and the right connecting part 364 are located on left and right sides of the crotch opening 34. The first U-shaped part 360, the left connecting part 363, the second U-shaped part 362 and the right connecting part 364 are sequentially connected to form a loop. The air inlet 370 is provided by the second U-shaped part 362, and the air outlet 371 is provided by the first U-shaped part 360. The airflow channel penetrates through the inside of the left connecting part 363 and/or the inside of the right connecting part 364. The first U-shaped part 360 is located at a front lower part of the connecting frame 3, and the first U-shaped part 360 surrounds a front side of the crotch opening 34. The second U-shaped part 362 is located at a rear upper part of the connecting frame 3, and the second U-shaped part 362 surrounds an upper side of the crotch opening 34. In a state where the connecting frame 3 is butted together with the working head 11, the first U-shaped part 360, the second U-shaped part 362, the left connecting part 363 and the right connecting part 364 are respectively attached to the working head 11.

The pants device 10 includes a pants main part 101 an a urine hood 107. The urine hood 107, the first U-shaped part 360, the left connecting part 363 and the right connecting part 364 are respectively bonded to the pants main part 101. The urine hood 107 is connected between the second U-shaped part 362 and the pants main part 101. A rear part of the urine hood 107 is bonded to the second U-shaped part 362. The first U-shaped part 360, the left connecting part 363 and the right connecting part 364 may be bonded to the pants main part 101 through strong glue.

In order to promote airflow diversion and inflow, the airflow channel includes a left side channel 372 communicating the air inlet 370 with the air outlet 371 and a right side channel 373 communicating the air inlet 370 with the air outlet 371. The left side channel 372 penetrates through the inside of the left connecting part 363, and the right side channel 373 penetrates through the inside of the right connecting part 364. The left connecting part 363 is hollow, and the internal space of the left connecting part 363 forms a part of the left side channel 372. The right connecting part 364 is hollow, and the internal space of the right connecting part 364 forms a part of the right side channel 373.

The second U-shaped part 362 includes a division part 390. The division part 390 divides the air inlet 370 into a left half part 3700 and a right half part 3701. The left side channel 372 correspondingly communicates the left half part 3700 of the air inlet 370 with the air outlet 371, and the right side channel 373 correspondingly communicates the right half part 3701 of the air inlet 370 and the air outlet 371. The division part 390 further promotes airflow diversion.

The connecting frame 3 is hollow so as to form the airflow channel inside the connecting frame 3, therefore, the airflow channel can also be referred to as an airflow cavity channel, the left side channel can also be referred to as a left side cavity channel, and the right side channel can also be referred to as a right side cavity channel. The connecting frame 3 includes a first frame body 380 and a second frame body 381. Each of the first frame body 380 and the second frame body 381 is one-piece component that is integrally formed. The first frame body 380 and the second frame body 381 are able to be made of plastic materials. The first frame body 380 and the second frame body 381 are respectively annular. The first frame body 380 and the second frame body 381 are combined together to form the connecting frame 3. The left half part of the first frame body 380 and the left half part of the second frame body 381 are combined together, and the left side channel 372 is formed between the left half part of the first frame body 380 and the left half part of the second frame body 381. The right half part of the first frame body 380 and the right half part of the second frame body 381 are combined together, and the right side channel 373 is formed between the right half part of the first frame body 380 and the right half part of the second frame body 381. In this embodiment, the division part 390 belongs to a part of the second frame body 381, a rear upper part of the first frame body 380 and a rear upper part of the second frame body 381 basically belong to the second U-shaped part 362, a front lower part of the first frame body 380 and a front lower part of the second frame body 381 basically belong to the first U-shaped part 360, the part connected between the rear upper part and front lower part of the first frame body 380 basically belongs to the left connecting part 363 and the right connecting part 364, and the part connected between the rear upper part and front lower part of the second frame body 381 also basically belongs to the left connecting part 363 and the right connecting part 364.

The pants device 10 includes a through hole 1070. The air inlet 370 is in fluid communication between the through hole 1070 and the airflow channel. The through hole 1070, the air inlet 370, the airflow channel and the air outlet 371 are in communication sequentially. When the air pressure at the air outlet 371 is lower than the air pressure at the through hole 1070, air sequentially flows through the through hole 1070, the air inlet 370, the airflow channel and the air outlet 371, so that the air at the through hole 1070 may be supplemented into the air outlet 371, and further flows to a specific object through the air outlet 371.

The pants device 10 includes a first U-shaped edge part 1071. The through hole 1070 is arranged at the first U-shaped edge part 1071. A part of the urine hood 107 (namely the first U-shaped edge part 1071) may be prevented from blocking the air inlet 370 by the through hole 1070. Therefore, in this embodiment, the through hole 1070 can also be referred to as an avoiding port. The through hole 1070, the air inlet 370, the left side channel 372 and the air outlet 371 are in communication sequentially to form an airflow path, and the through hole 1070, the air inlet 370, the right side channel 373 and the air outlet 371 are in communication sequentially to form another airflow path. By the two airflow paths, the diversion and inflow of the air during the flow process may be achieved. In this embodiment, the first U-shaped edge part 1071 belongs to a part of the urine hood 107 of the pants device 10, and the first U-shaped edge part 1071 is located at a rear part of the urine hood 107. The pants device 10 includes a first U-shaped edge part 1071, and the first U-shaped edge part 1071 is attached on the second U-shaped part 362. In this embodiment, the first U-shaped edge part 1071 spans a top side of the second U-shaped part 362, and the first U-shaped edge part 1071 is attached on the second U-shaped part 362.

The pants device 10 includes a second U-shaped edge part 1073 and a pants main part 101. The second U-shaped edge part 1073 is located on a front side of the second U-shaped part 362, and the second U-shaped edge part 1073 is attached on a front surface of the second U-shaped part 362. The second U-shaped edge part 1073 belongs to a part of the urine hood 107. The urine hood 107 is located between the second U-shaped part 362 and the pants main part 101. The urine hood 107 includes a third U-shaped edge part 1074, and the third U-shaped edge part 1074 is attached on the pants main part 101. The first U-shaped edge part 1071, the second U-shaped edge part 1073 and the third U-shaped edge part 1074 may be attached on the second U-shaped part 362 through strong glue. The urine hood 107 is made of a flexible material (such as silicone), so that all parts of the urine hood 107 can easily deform adaptively. The pants main part 101 includes a flexible buffer gasket 103 for attaching to the human body and a buttock pad 1080. The flexible buffer gasket 103 is configured for closely attaching to the human body to prevent excreta from leaking. The buttock pad 1080 is configured for attaching to human buttocks. The pants main part 101 includes an upper wrapping sheet 104, a lower wrapping sheet 105, and a connecting part 106 for connecting the upper wrapping sheet 104 with the lower wrapping sheet 105. The upper wrapping sheet 104 corresponds to the human abdomen, the lower wrapping sheet 105 corresponds to the human buttocks, the buttock pad 1080 is arranged on the lower wrapping sheet 105, the flexible buffer gasket 103 is arranged at the connecting part 106, and the connecting frame 3 is located on a back side of the connecting part 106. The upper wrapping sheet 104 and the lower wrapping sheet 105 respectively include magic tapes for connecting the upper wrapping sheet 104 with the lower wrapping sheet 105 to enable the pants device 10 to be tightly worn to the human body. The connecting part 106 is bonded to the connecting frame 3. The air inlet 370 is formed on the top side of the second U-shaped part 362, and the through hole 1070 is located above the air inlet 370 and is opposite to the air inlet 370 vertically.

The second frame body 381 is located between the pants main part 101 and the first frame body 380, and the airflow channel is formed between the first frame body 380 and the second frame body 381. In this embodiment, the first frame body 380 and the second frame body 381 are combined together along a front-and-rear direction, the first frame body 380 and the second frame body 381 are locked together by a plurality of screws, and the joint between the first frame body 380 and the second frame body 381 is sealed with glue to prevent the leakage of the airflow in the airflow channel through the joint.

The connecting frame 3 includes a slot 35, the slot 35 is located on a bottom side of the first U-shaped part 360, and a front end of the working head 11 may be forward inserted into the slot 35. The connecting frame 3 includes an arc-shaped retaining wall 361 for surrounding the slot 35. The arc-shaped retaining wall 361 is connected to the first U-shaped part 360. The slot 35 is formed between the first U-shaped part 360 and the arc-shaped retaining wall 361. In a state where the connecting frame 3 is butted together with the working head 11, a front end of the working head 11 is accommodated in the slot 35 and is attached to the arc-shaped retaining wall 361. In this embodiment, the arc-shaped retaining wall 361 belongs to a part of the second frame body 381. The second frame body 381 and the pants main part 101 are bonded together, and a frame opening provided and surrounded by the first frame body 380 and a frame opening provided and surrounded by the second frame body 381 are superposed together to form the crotch opening 34.

The air inlet 370 and the air outlet 371 are respectively formed between the first frame body 380 and the second frame body 381. A part of the first frame body 380 and a part of the second frame body 381 together form the air inlet 370, another part of the first frame body 380 and another part of the second frame body 381 together form the air outlet 371, and the air inlet 370 is higher than the air outlet 371. The air outlet 371 is located at a front end of the crotch opening 34. The air inlet 370 is formed on a top side of a rear upper end of the connecting frame 3, and the through hole 1070 is located above the air inlet 370. The highest point of the urine hood 107 is at least 1.5 cm higher than the through hole 1070. By this design, the highest point of the urine hood 107 may be pressed against the bedding and the like covered by the human body, thereby effectively inhibiting the obstruction of ventilation through the through hole 1070 caused by the bedding and the like covered by the human body pasting on the through hole 1070.

The connecting frame 3 is configured for connecting the pants device 10 with the working head 11. During use, the working head 11 is butted to the connecting frame 3 to achieve connection with the pants device 10. In a state where the connecting frame 3 is butted together with the working head 11, the connecting frame 3 is attached to the working head 11. The working head 11 in this embodiment is able to be pluggable forward and rearward relative to the pants device 10, and the working head 11 is able to be forward plugged to the connecting frame 3 of the pants device 10. During use, the working head 11 is forward plugged to the connecting frame 3 directly to achieve connection between the working head 11 and the pants device 10 very quickly and conveniently.

The working head 11 includes a left buckle member 12 located on a left side of the working head 11 and a right buckle member 13 located on a right side of the working head 11. The connecting frame 3 includes a left mating part 30 for snapping with the left buckle member 12 and a right mating part 31 for snapping with the right buckle member 13. In a state where the connecting frame 3 is butted together with the working head 11, the left buckle member 12 snaps with the left mating part 30, and the right buckle member 13 snaps with the right mating part 31. The left buckle member 12 includes a fastening protrusion 120, and the right buckle member 13 includes a fastening protrusion 130. The left mating part 30 includes a clamping groove and/or a convex rib for snapping with the fastening protrusion 120 of the left buckle member 12, and correspondingly, the right mating part 31 includes a clamping groove and/or a convex rib for snapping with the fastening protrusion 130 of the right buckle member 13. The left buckle member 12 and the right buckle member 13 are pivoted to a rear seat part 181 of the working head 11. During use, the working head 11 is forward butted to the connecting frame 3, the left buckle member 12 snaps with a left fastening part, and the right buckle member 13 snaps with a right fastening part, thereby connecting the pants device 10 with the working head 11 together. During unlocking, the left buckle member 12 and the right buckle member 13 may be pressed in opposite directions, so that the left buckle member 12 tilts leftward relative to the left mating part 30 so as to release the fastening fit from the left mating part 30, and the right buckle member 13 tilts rightward relative to the right mating part 31 so as to release the fastening fit from the right mating part 31.

The air inlet 370 is formed on a top side of a rear upper part of the connecting frame 3. The air outlet 371 is located at a front end of the crotch opening 34. The air outlet 371 is configured for supplementing air into the excretion recess 40. In a state where the connecting frame 3 is butted together with the working head 11, the air outlet 371 is adjacent to a front end of the excretion recess 40 and is located above the front end of the excretion recess 40. This design promotes effective supplementation of air into the excretion recess 40, thereby promoting more sufficient suction of the excreta collected by the excretion recess 40 rearward.

The working head 11 includes a front side nozzle 160 for spraying water. The front side nozzle 160 is located on a front side of the excretion recess 40. The front side nozzle 160 is configured for rearward spraying water into the excretion recess 40 to play a role in rearward flushing excreta in the excretion recess 40. In a state where the connecting frame 3 is butted together with the working head 11, the front side nozzle 160 is adjacent to the air outlet 371, and the front side nozzle 160 is located on a lower side of the air outlet 371. This arrangement enables the excreta collected by the excretion recess 40 to be rearward flushed to obtain more sufficient suction.

The air outlet 371 is located at an edge of the crotch opening 34, and the air outlet 371 is configured for discharging air to the excretion recess 40. In a state where the connecting frame 3 is butted together with the working head 11, the connecting frame 3 is attached to the working head 11, and the air inlet 370, the airflow channel, the air outlet 371 and the excretion recess 40 are in communication sequentially. Further, when the air pressure in the excretion recess 40 is lower than the air pressure outside the excrement collecting assembly (that is, when the air pressure in the excretion recess 40 is relative to the external negative pressure of the excrement collecting assembly), the air outside the excrement collecting assembly sequentially flows through the air inlet 370, the airflow channel and the air outlet 371 to supplement air into the excretion recess 40, so as to enable the excreta collected by the excretion recess 40 to be sucked more sufficiently.

The working head 11 includes a rear side nozzle 162 for spraying water, a front housing 4, and a drainage opening 43 for discharging excreta in the excretion recess 40. The rear side nozzle 162 is fixed on the front housing 4, and the front side nozzle 160 is also fixed on the front housing 4. The front housing 4 is concave to form the above excretion recess 40. The front housing 4 is a one-piece component that is integrally formed. The drainage opening 43 is arranged on the front housing 4, and the drainage opening 43 is located at a rear end of the excretion recess 40. The rear side nozzle 162 is located above the drainage opening 43, and the rear side nozzle 162 can play a role in washing the excretion part of the human body. In a state where the connecting frame 3 is butted together with the working head 11, the connecting frame 3 is attached to the front housing 4. More specifically, in a state where the connecting frame 3 is butted together with the working head 11, the first U-shaped part 360, second U-shaped part 362, left connecting part 363 and right connecting part 364 of the connecting frame 3 are respectively attached to the front housing 4. The front housing 4 includes a drying air outlet 371. The rear side nozzle 162 is located between the drainage opening 43 and the drying air outlet 371. The drying air outlet 371 is configured for drying the excretion part of the human body.

In a state where the connecting frame 3 is butted together with the working head 11, the air outlet 371 is adjacent to a front end of the excretion recess 40 and is located on an upper side of the front end of the excretion recess 40, and the air outlet 371 is configured for discharging air to the front end of the excretion recess 40. In a state where the connecting frame 3 is butted together with the working head 11, the urine hood 107 of the pants device 10 is located above the excretion recess 40, and the crotch opening 34 is butted together with the excretion recess 40, so that during human excretion, the excreta can enter the excretion recess 40 through the crotch opening 34.

The working head 11 has an attaching side 17 for attaching to the connecting frame 3. The excretion recess 40 is concave inward the working head 11 relative to the attaching side 17. In a state where the connecting frame 3 is butted together with the working head 11, the connecting frame 3 is attached to the attaching side 17 of the working head 11. The attaching side 17 includes a sealing ring 170. The connecting frame 3 and the attaching side 17 of the working head 11 may be sealed through the sealing ring 170 to avoid the leakage of excreta.

The working head 11 includes a base part 180 and a rear seat part 181. The excretion recess 40 is concavely arranged at the base part 180. The base part 180 extends forward from the rear seat part 181. The rear seat part 181 extends upward and is higher than the base part 180. In a state where the connecting frame 3 is butted together with the working head 11, the connecting frame 3 is attached to a top side of the base part 180 and a front side of the rear seat part 181. The top side of the base part 180 and the front side of the rear seat part 181 form the attaching side 17. A part of the front housing 4 forms a concave top wall of the base part 180, and another part of the front housing 4 forms a front wall of the rear seat part 181.

The pants device 10 includes a protective net 109. The protective net 109 is located between the through hole 1070 and the air inlet 370. The through hole 1070 is exposed outside the pants device 10. When the air pressure at the air outlet 371 is lower than the air pressure at the through hole 1070, the external air of the pants device 10 sequentially flows through the through hole 1070, the protective net 109, the air inlet 370, the airflow channel and the air outlet 371. The protective net 109 can prevent foreign matters from entering the air inlet 370 to a certain extent, thereby playing filtering and protecting roles. The protective net 109 is attached on the first U-shaped edge part 1071, and the protective net 109 completely covers the through hole 1070 from a downstream side of the through hole 1070 to enable the airflow flowing through the through hole 1070 to be filtered by the protective net 109, thereby enabling the protective net 109 to more effectively block foreign matters.

The disclosure further provides a nursing machine, including a host, the above excrement collecting assembly, a sewage suction pipe for connecting between the working head 11 and the host, a warm air pipeline for connecting between the working head 11 and the host, and a water supply pipeline for connecting between the working head 11 and the host. The host includes a negative pressure source for sucking excreta, a clean water bucket for providing cleaning water, a sewage bucket for storing the excreta sucked by the negative pressure source, a water pump for driving and delivering the water provided by the clean water bucket to the working head 11, and a plurality of wheels. The negative pressure source includes a motor and an impeller. The wheels are located at the bottom of the host to move the host. The impeller rotates under the drive of the motor to generate suction airflow so as to suck the excreta received by the excretion recess 40. Due to the suction effect of the negative pressure source, the air pressure in the excretion recess 40 is lower than the air pressure outside the excrement collecting assembly, and the excreta in the excretion recess 40 is sucked away. Specifically, during use, the pants device 10 is worn to the human body; under the suction effect of the negative pressure source, the air pressure at the air outlet 371 is lower than the air pressure at the air inlet 370 and the air pressure at the through hole 1070; from another perspective, under the suction effect of the negative pressure source, the air pressure in the excretion recess 40 is lower than the air pressure outside the excrement collecting assembly (that is, the air pressure in the excretion recess 40 is relative to the external negative pressure of the excrement collecting assembly); and due to the existence of pressure difference, the air outside the excrement collecting assembly sequentially flows through the air inlet 370, the airflow channel and the air outlet 371 to supplement air into the excretion recess 40, so as to enable the excreta collected by the excretion recess 40 to be sucked away more sufficiently. More specifically, under the suction of the negative pressure source of the host, the air pressure in the excretion recess 40 is lower than the air pressure outside the excrement collecting assembly, so that the air outside the excrement collecting assembly sequentially flows through the through hole 1070, the protective net 109, the air inlet 370, the airflow channel and the air outlet 371 and then is supplemented into the excretion recess 40. During use, the pants device 10 is worn to the human body; after human excretion, the negative pressure source sucks the excreta; and under the suction effect of the negative pressure source, the air pressure in the excretion recess 40 is lower than the air pressure outside the excrement collecting assembly, so that a part of the air outside the excrement collecting assembly sequentially flows through the through hole 1070, the protective net 109, the left half part 3700 of the air inlet 370, the left side channel 372 and the air outlet 371 and then is supplemented into the excretion recess 40, and another part of the air outside the excrement collecting assembly sequentially flows through the through hole 1070, the protective net 109, the right half part 3701 of the air inlet 370, the right side channel 373 and the air outlet 371 and then is supplemented into the excretion recess 40.

The working head 11 includes a drainage interface 190 and an adapting interface 191. The drainage interface 190 is connected to the sewage suction pipe. Under the suction effect of the negative pressure source, the excreta collected in the excretion recess 40 sequentially passes through the drainage opening 43, the drainage interface 190 and the sewage suction pipe and then is sucked into the host and stored in a sewage bucket of the host. The host delivers warm airflow to the working head 11 through the warm air pipeline, and the warm airflow is blown to the private part of the human body through the drying air outlet 371 so as to dry the private part of the human body. The host delivers clean water to the working head 11 through the water supply pipeline, and the clean water is sprayed out through the front side nozzle 160, the rear side nozzle 162 and the like.

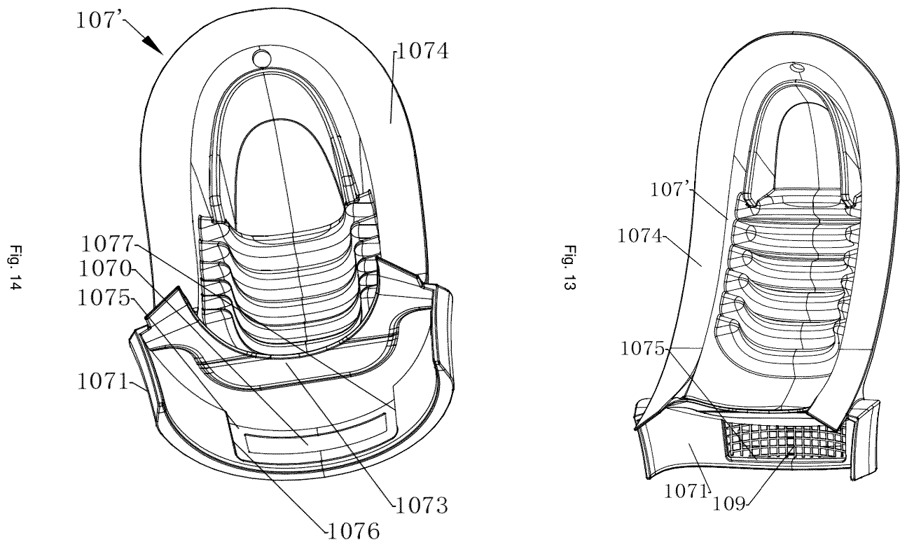

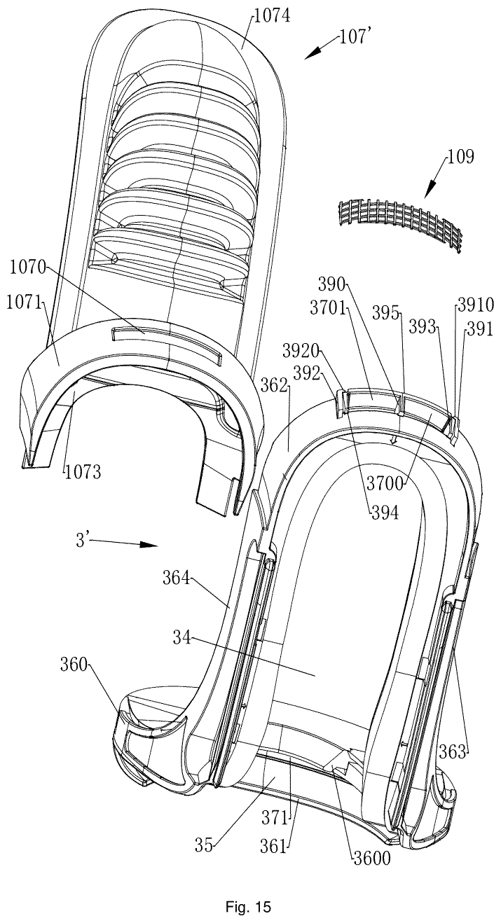

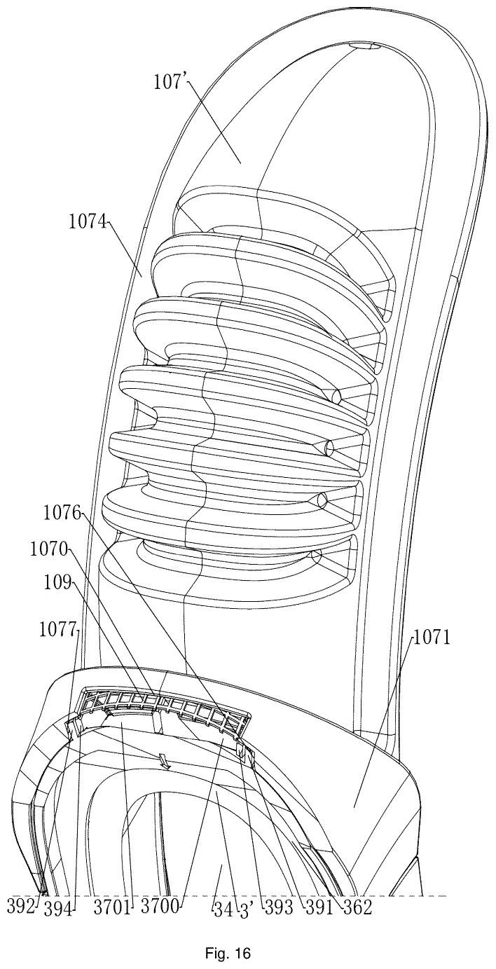

Referring to FIG. 13 to FIG. 16, the pants device 10 may also use another urine hood 107′ and another connecting frame 3′. The difference between the urine hood 107′ and the urine hood 107 is that the urine hood 107′ includes a concave region 1075. In addition to the concave region 1075, the urine hood 107′ is identical to the urine hood 107. For the urine hood 107′, more precisely, the first U-shaped edge part 1071 includes a concave region 1075. The difference between the connecting frame 3′ and the connecting frame 3 is that the connecting frame 3′ includes a first convex part 391, a second convex part 392, a third convex part 393, a fourth convex part 394, a connecting plate 395, and a baffle plate 3600. In addition to this, the connecting frame 3′ is identical to the connecting frame 3. For the connecting frame 3′, more precisely, the second U-shaped part 362 includes a first convex part 391, a second convex part 392, a third convex part 393, a fourth convex part 394, and a connecting plate 395, and the first U-shaped part 360 includes a baffle plate 3600.

The first convex part 391 and the second convex part 392 are at least partially accommodated in the concave region 1075 respectively, and the concave region 1075 is located directly above the second U-shaped part 362. The concave region 1075 is located between the through hole 1070 and the second U-shaped part 362, and the protective net 109 is accommodated in the concave region 1075. The through hole 1070 is directly in communication with the concave region 1075. The first convex part 391 and the second convex part 392 are embedded in the concave region 1075, the first convex part 391 is attached to a left side surface 1076 of the concave region 1075, and the second convex part 392 is attached to a right side surface 1077 of the concave region 1075. According to this design, when the first U-shaped edge part 1071 is placed on the second U-shaped part 362, the first convex part 391 and the second convex part 392 can limit the left and right deviation of the first U-shaped edge part 1071 to enable the first U-shaped edge part 1071 to be more easily placed on the second U-shaped part 362 in a certain place.

The first convex part 391 is located on a left side of the air inlet 370, and the second convex part 392 is located on a right side of the air inlet 370. The first convex part 391 and the second convex part 392 are located on a lower side of the protective net 109. An upper end of the first convex part 391 and an upper end of the second convex part 392 are in touch with the protective net 109. The third convex part 393 and the fourth convex part 394 are also located on left and right sides of the air inlet 370. The third convex part 393 and the fourth convex part 394 are embedded between the first convex part 391 and the second convex part 392. The third convex part 393 is correspondingly attached to the first convex part 391, and the fourth convex part 394 is correspondingly attached to the second convex part 392. The third convex part 393 is correspondingly snapped with the first convex part 391, and the fourth convex part 394 is correspondingly snapped with the second convex part 392. The first convex part 391 includes a first barb 3910 in fastening fit with the third convex part 393, and the second convex part 392 includes a second barb 3920 in fastening fit with the fourth convex part 394. Further, the third convex part 393 and the fourth convex part 394 are rearward embedded between the first convex part 391 and the second convex part 392. Moreover, the connecting plate 395 is located on a front side of the air inlet 370, a front surface of the connecting plate 395 belongs to a part of the front surface of the second U-shaped part 362 and is attached on the second U-shaped edge part 1073, the connecting plate 395 is at least partially accommodated in the concave region 1075, and the connecting plate 395 is integrally connected to the third convex part 393 and the fourth convex part 394.

The baffle plate 3600 blocks one side of the air outlet 371 to form the air outlet 371. The baffle plate 3600 is located at an edge of the crotch opening 34, and the baffle plate 3600 obliquely extends downward to limit the air discharge direction of the air outlet 371, so as to enable the air outlet 371 to obliquely discharge air downward. The first convex part 391 and the second convex part 392 respectively belong to the first frame body 380, and the third convex part 393, the fourth convex part 394, the connecting plate 395 and the baffle plate 3600 respectively belong to the second frame body 381. The edge of the part, adjacent to the baffle plate 3600, of the first frame body 380 and the baffle plate 3600 together form the air outlet 371.

In summary, the working head 11 may be detachably butted together with the pants device 10 to receive human excreta. The pants device 10 includes the connecting frame 3, and the pants device 10 is detachably butted together with the working head 11 by the connecting frame 3. The air outlet 371 is configured for supplementing air into the excretion recess 40, and when the air pressure in the excretion recess 40 is relative to the external negative pressure of the excrement collector, the air outside the excrement collector sequentially passes through the air inlet 370, the airflow channel and the air outlet 371 and then is supplemented into the excretion recess 40. When the air pressure in the excretion recess 40 is relative to the external negative pressure of the excrement collector, part of the air sequentially passes through the air inlet 370, the left side channel 372 and the air outlet 371 and then is supplemented into the excretion recess 40, and another part of the air sequentially passes through the air inlet 370, the right side channel 373 and the air outlet 371 and then is supplemented into the excretion recess 40. When the air pressure in the excretion recess 40 is relative to the external negative pressure of the excrement collector, the air outside the excrement collector sequentially passes through the through hole 1070, the air inlet 370, the airflow channel and the air outlet 371 and then is supplemented into the excretion recess 40. When the air pressure in the excretion recess 40 is relative to the external negative pressure of the excrement collector, part of the air sequentially passes through the through hole 1070, the air inlet 370, the left side channel 372 and the air outlet 371 and then is supplemented into the excretion recess 40, and another part of the air sequentially passes through the through hole 1070, the air inlet 370, the right side channel 373 and the air outlet 371 and then is supplemented into the excretion recess 40.

1. A pants device for wearing on a human body, comprising a connecting frame and a crotch opening for human excretion, wherein the connecting frame is located at a crotch of the pants device; the connecting frame is configured for connecting the pants device with a working head; the connecting frame surrounds at least a part of the crotch opening; the connecting frame comprises an air inlet, an air outlet, and an airflow channel; and the airflow channel communicates the air inlet with the air outlet.

2-35. (canceled)

36. An excrement collecting assembly, comprising the pants device for wearing on a human body and a working head, wherein the pants device comprises a connecting frame and a crotch opening for human excretion, wherein the connecting frame is located at a crotch of the pants device;
the connecting frame is configured for connecting the pants device with the working head, and the working head comprises an excretion recess for receiving human excreta;
the connecting frame surrounds at least a part of the crotch opening; the connecting frame comprises an air inlet, an air outlet, and an airflow channel; and the airflow channel communicates the air inlet with the air outlet.

37. The excrement collecting assembly according to claim 36, wherein the working head is able to be detachably butted together with the pants device, in a state where the connecting frame is butted together with the working head, the connecting frame is attached to the working head, and the air inlet, the airflow channel, the air outlet and the excretion recess are in communication sequentially.

38. The excrement collecting assembly according to claim 36, wherein the air outlet is for discharging air towards the excretion recess; the air outlet is located at an edge of the crotch opening; the pants device is able to be detachably butted together with the working head though the connecting frame; and when the air pressure in the excretion recess is lower than the air pressure outside the excrement collecting assembly, an air outside the excrement collecting assembly sequentially flows through the air inlet, the airflow channel and the air outlet to supplement air into the excretion recess.

39. (canceled)

40. The excrement collecting assembly according to claim 38, wherein
the airflow channel comprises a left side channel and a right side channel communicating the air inlet with the air outlet, the connecting frame further comprises a left connecting part and a right connecting part, the left connecting part is hollow, and the internal space of the left connecting part forms a part of the left side channel; and the right connecting part is hollow, and the internal space of the right connecting part forms a part of the right side channel;
the working head is pluggable forward and rearward relative to the pants device, and the working head may be forward plugged to the connecting frame of the pants device;
the working head comprises a left buckle member located on a left side of the working head and a right buckle member located on a right side of the working head; the connecting frame comprises a left mating part for snapping with the left buckle member and a right mating part for snapping with the right buckle member; and in a state where the connecting frame is butted together with the working head, the left buckle member snaps with the left mating part. and the right buckle member snaps with the right mating part.

41. (canceled)

42. The excrement collecting assembly according to claim 36, wherein the air inlet is formed on a top side of a rear upper part of the connecting frame; the air outlet is located at a front end of the crotch opening; the air outlet is configured for supplementing air into the excretion recess; and in a state where the connecting frame is butted together with the working head, the air outlet is adjacent to a front end of the excretion recess and is located above the front end of the excretion recess.

43. The excrement collecting assembly according to claim 36, wherein the working head comprises a front side nozzle for spraying water; the front side nozzle is located on a front side of the excretion recess; the front side nozzle is configured for rearward spraying water into the excretion recess; and in a state where the connecting frame is butted together with the working head, the front side nozzle is adjacent to the air outlet and is located on a lower side of the air outlet.

44. The excrement collecting assembly according to claim 43, wherein the working head further comprises a rear side nozzle for spraying water and a front housing; the rear side nozzle is fixed on the front housing; the front side nozzle is fixed on the front housing; the front housing is concave to form the excretion recess; the front housing is a one-piece component that is integrally formed; the front housing further comprises a drainage opening for discharging excreta in the excretion recess; the drainage opening is located at a rear end of the excretion recess; the rear side nozzle is located above the drainage opening; and in a state where the connecting frame is butted together with the working head, the connecting frame is attached to the front housing;
the front housing further comprises a drying air outlet, and the rear side nozzle is located between the drainage opening and the drying air outlet;
in a state where the connecting frame is butted together with the working head, the crotch opening is butted together with the excretion recess, and the urine hood of the pants device is located above the excretion recess;
the working head comprises a base part and a rear seat part; the excretion recess is concavely arranged at the base part; the base part extends forward from the rear seat part; the rear seat part extends upward and is higher than the base part; and in a state where the connecting frame is butted together with the working head, the connecting frame is attached to a top side of the base part and a front side of the rear seat part;
a part of the front housing forms a concave top wall of the base part, and another part of the front housing forms a front wall of the rear seat part.

45-48. (canceled)

49. A nursing machine, comprising an excrement collecting assembly, and the excrement collecting assembly comprises a pants device and a working head, the pants device for wearing on a human body comprises a connecting frame and a crotch opening for human excretion, wherein the connecting frame is located at a crotch of the pants device; the connecting frame is configured for connecting the pants device with the working head, and the working head comprises an excretion recess for receiving human excreta;
the connecting frame surrounds at least a part of the crotch opening; the connecting frame comprises an air inlet, an air outlet, and an airflow channel; and the airflow channel communicates the air inlet with the air outlet.

50. The excrement collecting assembly according to claim 36, wherein the connecting frame further comprises a first U-shaped part, a second U-shaped part, a left connecting part, and a right connecting part; the left connecting part and the right connecting part are located on left and right sides of the crotch opening; the first U-shaped part, the left connecting part, the second U-shaped part and the right connecting part are sequentially connected to form a loop; the air inlet is provided by the second U-shaped part, and the air outlet is provided by the first U-shaped part; and the airflow channel penetrates through the inside of the left connecting part and/or the inside of the right connecting part.

51. The excrement collecting assembly according to claim 50, wherein the first U-shaped part is located at a front lower part of the connecting frame, and the first U-shaped part surrounds a front side of the crotch opening; and the second U-shaped part is located at a rear upper part of the connecting frame, and the second U-shaped part surrounds an upper side of the crotch opening;
in a state where the connecting frame is butted together with the working head, the first U-shaped part, the second U-shaped part, the left connecting part and the right connecting part are respectively attached to the working head.

52. The excrement collecting assembly according to claim 50, wherein the airflow channel comprises a left side channel communicating the air inlet with the air outlet and a right side channel communicating the air inlet with the air outlet; the left side channel penetrates through the inside of the left connecting part; and the right side channel penetrates through the inside of the right connecting part.

53. The excrement collecting assembly according to claim 52, wherein the connecting frame comprises a first frame body and a second frame body; each of the first frame body and the second frame body is one-piece component that is integrally formed; the first frame body and the second frame body are respectively annular; the left half part of the first frame body and the left half part of the second frame body are combined together, and the left side channel is formed between the left half part of the first frame body and the left half part of the second frame body; and the right half part of the first frame body and the right half part of the second frame body are combined together, and the right side channel is formed between the right half part of the first frame body and the right half part of the second frame body.

54. The excrement collecting assembly according to claim 52, wherein the pants device further comprises an urine hood in a raised state, the urine hood comprises a first U-shaped edge part, wherein the first U-shaped edge part is attached on the second U-shaped part; the first U-shaped edge part is provided with a through hole; the through hole, the air inlet, the left side channel and the air outlet are in communication sequentially to form an airflow path; and the through hole, the air inlet, the right side channel and the air outlet are in communication sequentially to form another airflow path;
the left connecting part is hollow, and the internal space of the left connecting part forms a part of the left side channel; and the right connecting part is hollow, and the internal space of the right connecting part forms a part of the right side channel.

55. The excrement collecting assembly according to claim 36, wherein the connecting frame further comprises a first U-shaped part and a second U-shaped part, the air inlet is provided by the second U-shaped part, and the air outlet is provided by the first U-shaped part; the pants device further comprises an urine hood in a raised state, the urine hood comprises a first U-shaped edge part, wherein the first U-shaped edge part is attached on the second U-shaped part; the first U-shaped edge part is provided with a through hole; the air inlet is in fluid communication between the through hole and the airflow channel; and when the air pressure at the through hole is lower than the air pressure at the air inlet, air sequentially flows through the through hole, the air inlet, the airflow channel and the air outlet.

56. The excrement collecting assembly according to claim 55, wherein the first U-shaped edge part is attached on the second U-shaped part;
the first U-shaped edge part is located at a rear part of the urine hood;
the urine hood comprises a first U-shaped edge part, a second U-shaped edge part, wherein the second U-shaped edge part is located on a front side of the second U-shaped part, and the second U-shaped edge part is attached on a front surface of the second U-shaped part;
the air inlet is formed on the top side of the second U-shaped part, and the through hole is located above the air inlet and is opposite to the air inlet vertically.

57. The excrement collecting assembly according to claim 55, wherein the pants device further comprises a protective net, wherein the protective net is located between the through hole and the air inlet; and when the air pressure at the air outlet is lower than the air pressure at the through hole, the external air of the pants device sequentially flows through the through hole, the protective net, the air inlet, the airflow channel and the air outlet;
the first U-shaped edge part is attached on the second U-shaped part, and the protective net completely covers the through hole from an inner side of the through hole.

58. The excrement collecting assembly according to claim 36, wherein the connecting frame further comprises a first U-shaped part and a second U-shaped part, the air inlet is provided by the second U-shaped part, and the air outlet is provided by the first U-shaped part; the first U-shaped part further comprises a baffle plate for forming the air outlet; the baffle plate is located at an edge of the crotch opening; and the baffle plate obliquely extends downward to limit the air discharge direction of the air outlet so as to enable the air outlet to obliquely discharge air downward.

59. The excrement collecting assembly according to claim 36, wherein the connecting frame is annular; the connecting frame surrounds the crotch opening; the air inlet is located at the external periphery of the connecting frame, and the air outlet is located at the internal periphery of the connecting frame;
the connecting frame comprises a first frame body and a second frame body; the first frame body and the second frame body are combined together to form the connecting frame; the airflow channel is formed between the first frame body and the second frame body.

60. The excrement collecting assembly according to claim 36, wherein the pants device further comprises a protective net and an urine hood, the urine hood comprises a through hole, wherein the protective net is located between the through hole and the air inlet, and the through hole is exposed outside the pants device; and when the air pressure at the air outlet is lower than the air pressure at the through hole, the external air of the pants device sequentially flows through the through hole, the protective net, the air inlet, the airflow channel and the air outlet.
